(19) United States
(12) Patent Application Publication
(10) Pub. No.: US 2006/0120967 A1
(43) Pub. Date: Jun. 8, 2006

(54) Solution forms of cyclodextrins for nasal or throat delivery of essential oils

(75) Inventors: Ranga R. Namburi, Plainsboro, NJ (US); Sucharitha Jagini, Edison, NJ (US); Rama Prasad Karri, Plainsboro, NJ (US); Burgise F. Palkhiwala, East Windson, NJ (US)

Correspondence Address: Richard S. Roberts;Roberts & Roberts, LLP, P.O. Box 484, Princeton, NJ, 08542-0484, US

(73) Assignee: QPharma, LLC

(21) Appl. No.: 11/005,958

(22) Filed: Dec. 7, 2004

(51) Int. Cl. A61L 9/04; A61K 36/328; A61K 36/48; A61K 36/28; A61K 31/724

(52) U.S. Cl. 424/45; 514/58; 424/748; 424/757; 424/764; 424/641

(57) ABSTRACT

This invention further relates to a method for preventing or treating diseases or conditions of the oral cavity, throat or nose of warm-blooded animals including humans. More particularly, the invention pertains to a composition and method for spraying essential oils to the oral cavity, throat or nasal mucosa as cyclodextrin inclusion complexes. The spray composition includes a cyclodextrin in an amount of from about 0.1% w/v to about 20% w/v; at least one essential oil in an amount of from about 0.001% w/v to about 5.0% w/v; an effective amount of an antimicrobial preservative composition; and water. The composition may further comprise an alcohol co-solvent, a thickening agent, a sweetener, an antitussive, an anticholinergic, a decongestant, an antihistamine, an astringent, an anti-inflammatory steroid composition, a vitamin, a respiratory stimulant, a mucolytic agent, a bronchodilator, a beta-antagonist, an antidiarrheal agent, or combinations thereof.

### BACKGROUND OF THE INVENTION

1. Field of the Invention

The present invention pertains to aqueous, oral or nasal care solutions of essential oils suitable for oral spray or nasal spray administration to warm-blooded animals including humans. This invention further relates to a method for treating conditions or diseases of the oral cavity, throat or nose of warm-blooded animals including humans. More particularly, the invention pertains to a composition and method for administering essential oil containing compositions to the oral cavity, throat or nasal mucosa as cyclodextrin inclusion complexes. The invention composition may have enhanced duration of essential oil activity at the site of application due to gradual release of essential oil from the inclusion complexes. The composition may further comprise a a drug such as an antimicrobial agent, an antitussive, a decongestant, an anticholinergic, an antihistamine, an astringent, an anti-inflammatory steroid composition, a vitamin, a respiratory stimulant, a mucolytic agent, a bronchodilator, a beta-antagonist, an antidiarrheal agent, or combinations thereof. The composition may further comprise a flavoring agent, a flavor enhancing agent, a sweetener, a thickening agent and other formulation additives and combinations thereof.

2. Description of the Related Art

Essential oil compositions suitable for oral spray or nasal spray administration are known in the art. Commercially available drug products, for nasal decongestion, sinus relief, oral care compositions, decongestant throat spray, cough sprays, throat sprays for snore relief contain essential oils and or active pharmaceutical ingredients. Oily solutions of essential oils are unsuitable for use in nasal sprays as the vehicle inhibits ciliary movements and may cause lipoid pneumonia. Such essential oil compositions are difficult to formulate in aqueous solutions due to their poor solubility in water. Acceptable formulations must be able to dissolve an essential oil without precipitation or suspend the essential oil without agglomeration, precipitation or undue oxidation of the components, i.e. they must be stable. Suitable formulations must also avoid discomfort to the user.

Currently available compositions of essential oils, while safe and effective, use polysorbates (as solubilizing agents) and surfactants. Adjuvants such as alcohol (ethyl) are also used to make clear solutions of essential oils. Polysorbates, used in compositions for oral care application (mouth sprays) possess typical characteristic odor and some what bitter taste and when used in mouth application compositions produce unacceptable taste to the composition. Ethyl alcohol, at concentrations such as 20 percent or higher, causes irritation to the mucosa of the mouth. Adjuvants such as propylene glycol at concentration higher than 20 percent in nasal spray formulations produce stinging effect and causes discomfort at the time of use.

U.S. Pat. No. 6,689,342 and U.S. patent application publication 20040185010 relate to oral care compositions suitable for treating conditions of the oral cavity including a tropolone compound in combination with at least one essential oil, and a pharmaceutically acceptable oral carrier. No cyclodextrin inclusion complexes are shown. U.S. patent application publication 20040086539 shows a quick water-dissolving film containing aromatic, pharmaceutical or food substances including an essential oil, but again, no cyclodextrin inclusion complexes are shown. U.S. patent application publication 20040214797 shows preserved pharmaceutical compositions comprising cyclodextrins, but no essential oils are taught. In pharmaceutical formulations, cyclodextrins and cyclodextrin derivatives are often used to improve the solubility of a drug, see U.S. Pat. Nos. 5,089,482; 5,955,454; 5,089,482; U.S. Patent Application 2004022739; and WO 00/21503; however it has not been known to improve the solubility of essential oils by means of a cyclodextrin inclusion complex. Hence, there is a clear need for development of stable solution formulations of essential oils when used alone or in combination with active pharmaceutical ingredients for oral care in the form of mouth sprays or nasal care applications. The compositions of the present invention are stable, preservable, and are suitable for oral spray or nasal spray administration of essential oil containing compositions and have a acceptable mouth feel when sprayed in to mouth and have reduced stinging tendency when sprayed in to nasal cavity.

### SUMMARY OF THE INVENTION

The invention provides a composition suitable for oral spray or intranasal spray administration which comprises: a cyclodextrin in an amount of from about 0.1% to about 20% (w/v); at least one essential oil in an amount of from about 0.001% to about 5.0% (w/v); an effective amount of an antimicrobial preservative composition; and water.

The invention also provides a method of oral or nasal care which comprises orally spraying or intranasally spraying to a subject in need of oral or nasal care a composition which comprises: a cyclodextrin in an amount of from about 0.1% to about 20% (w/v); at least one essential oil in an amount of from about 0.001% to about 5.0% (w/v); an effective amount of an antimicrobial preservative composition; and water.

The composition may further comprising an alcohol co-solvent, a sweetener, a mucoadhesive thickening agent, a flavoring agent, and a flavor enhancing agent. The composition may further contain a pharmaceutical active agent such as an antitussive, a decongestant, an anticholinergic, an antihistamine, an astringent, an anti-inflammatory steroid composition, a vitamin, a respiratory stimulant, a mucolytic agent, a bronchodilator, a beta-agonist, an antidiarrheal, or combinations thereof. Commercially available container closure systems are used for filling of the invention formulations and appropriate metered dose pumps to deliver the spray orally or nasally.

### DETAILED DESCRIPTION OF THE INVENTION

The composition of the present invention includes a cyclodextrin. Cyclodextrins are a group of structurally related saccharides which are formed by enzymatic cyclization of starch by a group of amylases termed glycosyltransferases. Cyclodextrins are cyclic oligosaccharides, consisting of (α-1,4)-linked α-D-glucopyranose units, with a somewhat lipophilic central cavity and a hydrophilic outer surface. The most common naturally occurring cyclodextrins are α-cyclodextrin, β-cyclodextrin and γ-cyclodextrin consisting of 6, 7 and 8 glucopyranose units, respectively. Of these three derivatives, β-cyclodextrin appears to be the most useful pharmaceutical complexing agent due to its cavity size, availability, low cost and other properties. Cyclodextrin derivatives of current pharmaceutical interest include the hydroxypropyl derivatives of α-, β- and γ-cyclodextrin, sulfoalkylether cyclodextrins such as sulfobutylether β-cyclodextrin, alkylated cyclodextrins such as the randomly methylated β-cyclodextrin, and various branched cyclodextrins such as glucosyl- and maltosyl β-cyclodextrin.

In aqueous solutions, cyclodextrins form inclusion complexes with essential oils and many drugs through a process in which the water molecules located in the central cavity are replaced by either the whole drug molecule, or more frequently, by some lipophilic portion of the drug structure. Once included in the cyclodextrin cavity, the essential oil and drug molecules may be dissociated through complex dilution, by replacement of the included essential oil and drug by some other suitable molecule or, the drug may be transferred to the matrix for which it has the highest affinity. Importantly, since no covalent bonds are formed or broken during the drug-cyclodextrin complex formation, the complexes are in dynamic equilibrium with free essential oil and drug and cyclodextrin molecules. Useful cyclodextrins for use in the present invention non-exclusively include alkyl cyclodextrins, hydroxy alkyl cyclodextrin, such as hydroxy propyl β-cyclodextrin, carboxy alkyl cyclodextrins and sulfoalkyl ether cyclodextrin, such as sulfo butyl ether β-cyclodextrin. Examples of suitable cyclodextrins for use in the present invention non-exclusively include α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof. In one embodiment, the cyclodextrin may be present in the overall composition in an amount of from about 0.1% to about 20% w/v. In another embodiment, the cyclodextrin may be present in the overall composition in an amount of from about 0.5% to about 5% w/v. In yet another embodiment, the cyclodextrin may be present in the overall composition in an amount of from about 1.0% to about 2.5% w/v.

The composition of the present invention includes at least one essential oil. Useful essential oils non-exclusively include cineol (eucalyptol), thymol, menthol, methyl salicylate, wintergreen oil, carvacrol, camphor, anethole, carvone, eugenol, isoeugenol, limonene, osimen, n-decyl alcohol, citronel, α-salpineol, methyl acetate, citronellyl acetate, methyl eugenol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, star anise, or combinations thereof. In one embodiment, the essential oil component may be present in the overall composition in an amount of from about 0.001% to about 5.0% (w/v). In another embodiment, the essential oil component may be present in the overall composition in an amount of from about 0.01% (w/v) % to about 1.0% w/v. In yet another embodiment, the essential oil component may be present in the overall composition in an amount of from about 0.02 (w/v) % to about 0.5% w/v.

The composition of the present invention includes an effective amount of an antimicrobial preservative. Preservatives can be used to inhibit microbial growth in the compositions. An “effective amount” of a preservative is that amount necessary to prevent the growth of microorganisms in the composition. The amount of preservative is generally that which is necessary to prevent microbial growth in the composition for a storage period of at least six months. Examples of pharmaceutically acceptable preservatives non-exclusively include cetyl pyridinium chloride, phenol, benzethonium chloride, butylparaben, methyl paraben, ethyl paraben, propyl paraben, benzalkonium chloride, thimerosal, chlorobutanol, phenylethyl alcohol, benzyl alcohol, potassium sorbate, sodium benzoate, sorbic acid or combinations thereof. In one embodiment, the antimicrobial preservative may be present in the composition in an amount of from about 0.002% to about 1.0% w/v. In another embodiment, the antimicrobial preservative may be present in the composition in an amount of from about 0.005% to about 0.1% w/v. In yet another embodiment, the antibicrobial preservative may be present in the composition in an amount of from about 0.01% to about 0.05% w/v.

The composition of the present invention then comprises sufficient water to make-up the composition in the desired dosage. Preferably the water is pharmaceutical quality purified water. In one embodiment, the purified water may be present in the composition in an amount of from about 65.0% to about 98.0% by volume. In another embodiment, the purified water may be present in the composition in an amount of from about 90.0% to about 96% by volume. In yet another embodiment, the purified water may be present in the composition in an amount of from about 93.0% to about 95.5% by volume.

The composition may optionally further comprise an alcohol co-solvent. Useful co-solvent alcohols non-exclusively include propylene glycol, ethyl alcohol, butyl alcohol, glycerin, hexylene glycol, isopropyl alcohol, polyethylene glycol, polyhydric alcohols, or combinations thereof. Polyhydric alcohols are preferred as co-solvents and propylene glycol is most preferred. In one embodiment, the alcohol co-solvent may be present in the composition in an amount of from about 0.2% w/v to about 35% w/v. In another embodiment, the cosolvent may be present in the composition in an amount of from about 0.2% w/v to about 10.0% w/v. In still another embodiment the cosolvent may be present in the composition in an amount of from about 1.0% to about 10.0% w/v. In yet another embodiment, the cosolvent may be present in the composition in an amount of from about 2.0% to about 5.0% w/v.

The composition may optionally further comprise a sweetener. Useful sweeteners non-exclusively include glucose, fructose, xylitol, sucralose, saccharin, aspartame, acesulfame potassium, cyclamate, neohesperidine DC, thaumatin, alitame, stevioside, or combinations thereof. In one embodiment, the sweetener may be present in the composition in an amount of from about 0.01% to about 25% w/v. In another embodiment, the sweetener may be present in the composition in an amount of from about 0.1% % to about 10.0% w/v. In still another embodiment the sweetener may be present in the composition in an amount of from about 0.2% to about 5.0% w/v.

The composition may optionally further comprise a mucoadhesive polymer thickening agent to achieve a longer residence time in the mouth and as also in the nose. Such mucoadhesive polymers avoid dripping of spray liquid in the nose. Useful thickening agents non-exclusively include hydroxy alkyl alky celluloses such as hydroxy propyl methyl cellulose, hydroxylethyl cellulose, hydroxyl methyl cellulose; carboxy alkyl celluloses and their salts such as sodium carboxy methyl cellulose; methyl cellulose; polysaccharides such as alginic acid, agar agar, guar gum, xanthan gum; polyacrylic acids such as polymethacrylic acid derivatives; polyvinyl pyrrolidone, maltodextrines. The thickening agent may be present in the composition in an amount of from about 0.05% to about 10%. In another embodiment, the thickening agent may be present in the composition in an amount of from about 0.1% to about 5.0% w/v. In still another embodiment the thickening agent may be present in the composition in an amount of from about 0.2% to about 2.0% w/v.

The composition may optionally further comprise a flavoring and or flavor enhancing agents. Useful flavoring agents non-exclusively include chocolate, cherry, strawberry, raspberry, root beer and others, or combinations thereof. The flavoring agent may be present in the composition in an amount of from about 0.05% w/v. to about 5.0% w/v. In another embodiment, the flavor may be present in the composition in an amount of from about 0.1% w/v to about 2.0% w/v. In still another embodiment the flavor may be present in the composition in an amount of from 0.2% w/v to about 1.0% w/v.

The composition may optionally further comprise an antitussive. Useful antitussives non-exclusively include codeine, hydrocodone, dextromethorphan, dextromethorphan HBr, a demulcent, an expectorant, or combinations thereof. The antitussive may be present in the composition in an amount of from about 0.1% to about 10.0% w/v. In another embodiment, the antitussive may be present in the composition in an amount of from about 0.5% to about 7.5% w/v. In still another embodiment the antitussive may be present in the composition in an amount of from about 1.0% to about 5.0% w/v.

The composition may optionally further comprise an anticholinergic. Useful anticholinergics non-exclusively include atropine, scopolamine, methscopolamine nitrate, glycopyrrolate, benztropine, trihexyphenidyl, or combinations thereof. The anticholinergic may be present in the composition in an amount of from about 0.01% to about 10.0% w/v. In another embodiment, the anticholinergic may be present in the composition in an amount of from about 0.1% to about 7.5% w/v. In still another embodiment the anticholinergic may be present in the composition in an amount of from about 0.2% to about 5.0% w/v.

The composition may optionally further comprise a decongestant. Useful decongestants non-exclusively include oxymetazoline HCl, phenylephrine HCl, chlorpheniramine, pseudoephedrine hydrochloride, or combinations thereof. The decongestant may be present in the composition in an amount of from about about 0.1% to about 10.0% w/v. In another embodiment, the decongestant may be present in the composition in an amount of from about 0.5% to about 7.5% w/v. In still another embodiment the decongestant may be present in the composition in an amount of from about 1.0% to about 5.0% w/v.

The composition may optionally further comprises an antihistamine. Useful antihistamines non-exclusively include loratidine, fexofenadine, chlorpheniramine, chlorpheniramine maleate, cyproheptadine, doxylamine, hydroxyzine, dimenhydrinate, diphenhydramine, doxylamine, meclizine, promethazine, or combinations thereof. The antihistamine may be present in the composition in an amount of from about 0.1% to about 10.0% w/v. In another embodiment, the antihistamine may be present in the composition in an amount of from about 0.25% to about 5.0% w/v. In still another embodiment the antihistamine may be present in the composition in an amount of from about 1.0% to about 4.0% w/v.

The composition may optionally further comprise an astringent. Useful astringent, non-exclusively include zinc gluconate, zinc chloride, zinc acetate, myrrh, acacia, black catechu, carum copticum, tannin, rhatany, alum or combinations thereof. The astringent may be present in the composition in an amount of from about 0.1% to about 10.0% w/v. In another embodiment, the astringent may be present in the composition in an amount of from about 0.5% to about 7.5% w/v. In still another embodiment the astringent may be present in the composition in an amount of from about 1.0% to about 5.0% w/v.

The composition may optionally further comprise an anti-inflammatory steroid, such as a corticosteroid. The corticosteroids that are useful in the present invention generally include any steroid produced by the adrenocortex, including glucocorticoids and mineralocorticoids, and synthetic analogs and derivatives of naturally occurring corticosteroids having anti-inflammatory activity. Examples of corticosteroids that can be used in the compositions of the invention include aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, fluticasone propionate, halcinonide, hydrocortisone, icomethasone, meprednisone, methylprednisolone, mometasone paramethasone, mometasone furoate monohydrate, prednisolone, prednisone, tixocortol, triamcinolone, and their respective pharmaceutically acceptable derivatives, such as beclomethasone diproprionate, dexamethasone 21-isonicotinate, icomethasone enbutate, tixocortol 21-pivalate, and triamcinolone acetonide. Particularly preferred are compounds such as beclomethasone diproprionate, budesonide, flunisolide, fluticasone propionate, mometasone and triamcinolone acetonide. In one embodiment, the steroid may be present in the anti-inflammatory steroid composition in an amount of from about 0.0001% to about 2.0% w/v. In another embodiment, the steroid may be present in the anti-inflammatory steroid composition in an amount of from about 0.0005% to about 1.0% w/v. In yet another embodiment, the steroid may be present in the anti-inflammatory steroid composition in an amount of from about 0.001% to about 0.1% w/v.

The composition may optionally further comprise a vitamin. Useful vitamins, non-exclusively include Vitamin A, Vitamin B₁, Vitamin B₂, Vitamin B₆, Vitamin B₁₂, folic Acid, Vitamin C, Vitamin D, Vitamin E, Vitamin H, Vitamin K, Vitamin P, or combinations. The vitamin component may be present in the composition in an amount of from about 0.001% to about 10.0% w/v. In another embodiment, the vitamin component may be present in the composition in an amount of from about 0.01% to about 5.0% w/v. In still another embodiment the vitamin component may be present in the composition in an amount of from about 0.1% to about 2.5% w/v.

The composition may optionally further comprise a respiratory stimulant. Useful respiratory stimulants non-exclusively include progesterone, protriptyline HCl, naloxone HCl, almitrine, doxapram, theophylline, or combinations thereof. The respiratory stimulant may be present in the composition in an amount of from about 0.1% to about 10.0% w/v. In another embodiment, the respiratory stimulant may be present in the composition in an amount of from about 0.5% to about 7.5% w/v. In still another embodiment the respiratory stimulant may be present in the composition in an amount of from about 1.0% to about 5.0% w/v.

The composition may optionally further comprise a mucolytic agent. Useful mucolytic agents non-exclusively include trypsin, sodium bicarbonate, acetylcysteine, guaifenesin, or combinations thereof. The mucolytic agent may be present in the composition in an amount of from about 0.1% to about 10.0% w/v. In another embodiment, the mucolytic agent may be present in the composition in an amount of from about 0.5% to about 7.5% w/v. In still another embodiment the mucolytic agent may be present in the composition in an amount of from about 1.0% to about 5.0% w/v.

The composition may optionally further comprise a bronchodilator. Useful bronchodilators non-exclusively include albuterol, albuterol sulfate, epinephrine, ipratropium, isoetharine, isoproterenol, levalbuterol HCl, metaproterenol, pirbuterol acetate, terbutalene, theophylline, or combinations thereof. The bronchodilator may be present in the composition in an amount of from about 0.1% to about 10.0% w/v. In another embodiment, the bronchodilator may be present in the composition in an amount of from about 0.5% to about 7.5% w/v. In still another embodiment the bronchodilator may be present in the composition in an amount of from about 1.0% to about 5.0% w/v.

The composition may optionally further comprise a beta-antagonist. Useful beta-antagonists non-exclusively include fenoterol, metaproterenol, procaterol, salbutamol, terbutaline or combinations thereof. The beta-antagonist may be present in the composition in an amount of from about 0.1% to about 10.0% w/v. In another embodiment, the beta-antagonist may be present in the composition in an amount of from about 0.2% to about 5.0% w/v. In still another embodiment the beta-antagonist may be present in the composition in an amount of from about 0.25% to about 2.5% w/v.

The composition may optionally further comprise an antidiarrheal agent. Useful antidiarrheals non-exclusively include loperamide hydrochloride, attapulgite, Bismuth subsalicylate or combinations thereof. The antidiarrheal may be present in the composition in an amount of from about 0.1% to about 10.0% w/v. In another embodiment, the antidiarrheal may be present in the composition in an amount of from about 0.5% to about 7.5% w/v. In still another embodiment the antidiarrheal may be present in the composition in an amount of from about 1.0% to about 2.5% w/v.

The complexes are usually prepared by addition of required amount of the essential oil in to water containing cyclodextrin and stirring the mixture until all of the essential oil gets in to solution to form a clear liquid. Optional adjuvants such as polyhydric alcohols could be used to make initial solution of essential oil and adding this combination to water containing cyclodextrin under stirring. Aqueous solutions of active pharmaceutical ingredients or other formulation ingredients are incorporated in to the above clear solution to make the final formulation. A useful pH range for a nasal formulations is from about 4.5 to about 7.0. For oral applications a useful pH range is from about 4.0 to about 7.5. The pH may be adjusted with dilute hydrochloric acid or dilute sodium hydroxide.

The finished formulation is filtered through appropriate filter and the compositions are filled into commercially available bottles and fit with metered dose pumps for oral or nasal delivery of the drug products. Commercially available metering pumps for oral or nasal route application are used to deliver the appropriate dose of composition per actuation. Such are available from Valois Pharmaceutical Division, Pfeiffer of America, and Saint-Gobain Calmar, Inc. The delivery dose volumes of metered pumps may vary from about 25 microliters to about 300 microliters.

The present invention is explained in greater detail in the following non-limiting examples.

### EXAMPLE 1

Oral Decongestant Spray Composition:

This example describes the preparation of an oral decongestant spray composition in accordance with the invention, which is useful for alleviation of congestion in the throat and nose. Ingredients for the preparation of the oral decongestant spray solution are set forth in the table below.

| Ingredient | % quantity | per 500 mL | |
|---|---|---|---|
| Phenylephrine HCl | 5.0 | 25.0 | g |
| Cetyl Pyridinium Chloride | 0.05 | 0.25 | g |
| Sucralose 25% conce. | 0.5 | 2.5 | g |
| Xylitol | 5.0 | 25.0 | g |
| Glycerin | 5.0 | 25.0 | g |
| Cineol (eucalyptus oil) | 0.25 | 1.25 | g |
| Menthol | 0.1 | 0.5 | g |
| Wintergreen oil | 0.03 | 0.15 | g |
| Hydroxypropyl β Cyclodextrin | 2.0 | 10.0 | g |
| Chocolate flavor | 0.5 | 2.5 | g |

| | | |
|---|---|---|
| Purified Water | qs | qs |

Process:

Take 350 g of purified water in an appropriate container. Add and dissolve hydroxy propyl β Cyclodextrin under stirring. To this mixture add cineole and winter green oil under stirring and continue stirring until it forms a clear solution. Separately take glycerin and dissolve menthol with slight application of heat (heated to no more than 35° C.) and add this to the main bulk. Make aqueous solutions of other ingredients taking small quantities of water and incorporate in to the main bulk solution under stirring. Incorporate flavor under stirring and check for clarity, pH and make up the volume before filtering the solution through 0.45 micron membrane filter. Fill the liquid in appropriate container closure systems fit with metered dose pump.

### EXAMPLE 2

Nasal Decongestant and Sinusitis Relief Spray Composition:

This example describes the preparation of a nasal decongestant and sinusitis relief spray composition in accordance with the invention, which is useful for alleviation of congestion and relief from sinusitis in the nose. Ingredients for the preparation of the nasal decongestant and sinusitis relief spray solution are set forth in the table below.

| Ingredient | % quantity | per 500 mL | |
|---|---|---|---|
| Oxymetazoline HCl | 0.05 | 0.25 | g |
| Cetyl Pyridinium Chloride | 0.05 | 0.25 | g |
| Camphor | 0.02 | 0.1 | g |
| Cineol (eucalyptus oil) | 0.05 | 0.25 | g |
| Menthol | 0.05 | 0.25 | g |
| Edetate Disodium | 0.02 | 0.1 | g |
| Propylene Glycol | 2.0 | 10.0 | g |
| Sodium Phosphate dibasic | 0.005 | 0.025 | g |
| Sodium Phosphate monobasic | 0.2 | 1.0 | g |
| Hydroxypropyl β Cyclodextrin | 2.0 | 10.0 | g |

| | | |
|---|---|---|
| Purified Water | Qs | qs |

Process:

Take 400 g of purified water in an appropriate container. Add and dissolve hydroxy propyl β Cyclodextrin under stirring. To the Cyclodextrin solution add cineole, camphor and winter green oil mixture under stirring and continue stirring until a clear solution is obtained. Separately take propylene glycol and dissolve menthol with slight application of heat (heated to no more than 35° C.) and add this to the main bulk. Make aqueous solutions of other ingredients taking small quantities of water and incorporate in to the main bulk solution under stirring. Check for clarity, pH and make up the volume before filtering the solution through 0.45 micron membrane filter. Fill the liquid in appropriate container closure systems fit with metered dose pump for nasal delivery.

### EXAMPLE 3

Sore Throat Spray Composition:

This example describes the preparation of a Throat spray composition in accordance with the invention, which is useful for alleviation of soreness in the throat. Ingredients for the preparation of the sore throat spray solution are set forth in the table below.

| Ingredient | % quantity | per 200 mL | |
|---|---|---|---|
| Cetyl Pyridinium Chloride | 0.05 | 0.1 | g |
| Benzalkonium Chloride | 0.02 | 0.04 | g |
| Edetate Disodium | 0.02 | 0.04 | g |
| Glycerin | 5.0 | 10.0 | g |
| Cineol (eucalyptus oil) | 0.25 | 0.5 | g |
| Menthol | 0.25 | 0.5 | g |
| Hydroxypropyl β Cyclodextrin | 3.0 | 6.0 | g |
| Xylitol | 5.0 | 10.0 | g |
| Sucralose 20% Conc. | 0.5 | 1.0 | g |
| Zinc Acetate | 0.05 | 0.1 | g |

| | | |
|---|---|---|
| Purified Water | Qs | qs |

Process:

Take 150 g of purified water in an appropriate container. Add and dissolve hydroxy propyl β Cyclodextrin under stirring. Add cineole to Cyclodextrin solution under stirring to make a clear solution. Separately take glycerin and dissolve menthol with slight application of heat (heated to no more than 35° C.) and add this to the main bulk. Make aqueous solutions of other ingredients taking small quantities of water and incorporate in to the main bulk solution under stirring. Check for clarity, pH and make up the volume before filtering the solution through 0.45 micron membrane filter. Fill the liquid in appropriate container closure systems fit with metered dose pump.

### EXAMPLE 4

Anti-Snore Composition:

This example describes the preparation of a mouth spray composition in accordance with the invention, which is useful to stop snoring. Ingredients for the preparation of the Anti Snore spray solution are set forth in the table below.

| Ingredient | % quantity | per 500 mL | |
|---|---|---|---|
| Cetyl Pyridinium Chloride | 0.05 | 0.25 | g |
| Sodium Saccharin | 0.03 | 0.15 | g |
| Glycerin | 5.0 | 25.0 | g |
| Cineol (eucalyptus oil) | 0.25 | 1.25 | g |
| Menthol | 0.1 | 0.5 | g |
| Peppermint oil | 0.02 | 0.1 | g |
| Wintergreen oil | 0.03 | 0.15 | g |
| Hydroxypropyl β Cyclodextrin | 2.0 | 10.0 | g |

| | | |
|---|---|---|
| Purified Water | qs | qs |

Process:

Take 400 g of purified water in an appropriate container. Add and dissolve hydroxy propyl β Cyclodextrin under stirring. Add cineole, peppermint oil and wintergreen oil to Cyclodextrin solution under stirring to make a clear solution. Separately take glycerin and dissolve menthol with slight application of heat (heated to no more than 35° C.) and add this to the main bulk. Dissolve sodium saccharin in purified water and add it to the main bulk under stirring. Check for clarity, pH and make up the volume before filtering the solution through 0.45 micron membrane filter. Fill the liquid in appropriate container closure systems fit with metered dose pump.

### EXAMPLE 5

Tea Tree Oil with other Essential Oils Spray Composition:

This example describes the preparation of an oral and nasal care spray composition in accordance with the invention. This composition containing Tea Tree Oil possesses good antimicrobial properties for both oral and nasal care applications. Ingredients for the preparation of the oral and nasal care solution are set forth in the table below.

| Ingredient | % quantity | per 500 mL | |
|---|---|---|---|
| Cetyl Pyridinium Chloride | 0.05 | 0.25 | g |
| Sucralose 25% conce. | 0.25 | 1.25 | g |
| Glycerin | 5.0 | 25.0 | g |
| Tea Tree Oil | 0.25 | 1.25 | g |
| Cineol (eucalyptus oil) | 0.25 | 1.25 | g |
| Menthol | 0.05 | 0.25 | g |
| Wintergreen oil | 0.03 | 0.15 | g |
| Hydroxypropyl β Cyclodextrin | 2.0 | 10.0 | g |

| | | |
|---|---|---|
| Purified Water | qs | qs |

Process:

Take 350 g of purified water in an appropriate container. Add and dissolve hydroxy propyl β Cyclodextrin under stirring. To this mixture add tea tree oil, cineole, and winter green oil under stirring and continue stirring until it forms a clear solution. Separately take glycerin and dissolve menthol with slight application of heat (heated to no more than 35° C.) and add this to the main bulk. Incorporate sucralose conc. in to the main bulk under stirring. Check for clarity and pH before and make up the volume before filtering the solution through 0.45 micron membrane filter. Fill the liquid in appropriate container closure systems fit with metered dose pump.

### EXAMPLE 6

Oral Decongestant with Anti-histaminic Spray Composition:

This example describes the preparation of an oral decongestant with an antihistamine oral spray composition in accordance with the invention, which is useful for alleviation of congestion in the throat and relief of cold related allergies. Ingredients for the preparation of the oral decongestant and anti-histaminic spray solution are set forth in the table below.

| Ingredient | % quantity | per 500 mL | |
|---|---|---|---|
| Phenylephrine HCl | 5.0 | 25.0 | g |
| Chlorpheniramine Maleate | 2.0 | 10.0 | g |
| Cetyl Pyridinium Chloride | 0.05 | 0.25 | g |
| Sucralose 25% conce. | 0.5 | 2.5 | g |
| Glycerin | 5.0 | 25.0 | g |
| Cineol (eucalyptus oil) | 0.25 | 1.25 | g |
| Menthol | 0.1 | 0.5 | g |
| Wintergreen oil | 0.03 | 0.15 | g |
| Hydroxypropyl β Cyclodextrin | 2.0 | 10.0 | g |
| Cherry flavor | 0.25 | 1.25 | g |

| | | |
|---|---|---|
| Purified Water | qs | qs |

Process:

Take 300 g of purified water in an appropriate container. Add and dissolve hydroxy propyl β Cyclodextrin under stirring. To this mixture add cineole and winter green oil under stirring and continue stirring until it forms a clear solution. Separately take glycerin and dissolve menthol with slight application of heat (heated to no more than 35° C.) and add this to the main bulk. Make aqueous solutions of other ingredients taking small quantities of water and incorporate in to the main bulk solution under stirring. Incorporate flavor under stirring and check for clarity, pH and make up the volume before filtering the solution through 0.45 micron membrane filter. Fill the liquid in appropriate container closure systems fit with metered dose pump.

### EXAMPLE 7

Oral Decongestant/Anti-histaminic/Anti-muscarinic Spray Composition:

This example describes the preparation of an oral decongestant/antihistamine/antimuscarinic oral spray composition in accordance with the invention, which is useful for alleviation of respiratory congestion, allergic rhinitis and vasomotor rhinitis. Ingredients for the preparation of the oral decongestant/anti-histaminic/antimuscarinic spray solution are set forth in the table below.

| Ingredient | % quantity | per 500 mL | |
|---|---|---|---|
| Phenylephrine HCl | 5.0 | 25.0 | g |
| Chlorpheniramine Maleate | 1.0 | 5.0 | g |
| Methscopolamine Nitrate | 0.625 | 3.125 | g |
| Cetyl Pyridinium Chloride | 0.05 | 0.25 | g |
| Sucralose 25% conce. | 0.5 | 2.5 | g |
| Glycerin | 5.0 | 25.0 | g |
| Cineol (eucalyptus oil) | 0.25 | 1.25 | g |
| Menthol | 0.1 | 0.5 | g |
| Wintergreen oil | 0.03 | 0.15 | g |
| Hydroxypropyl β Cyclodextrin | 2.0 | 10.0 | g |
| Root Beer flavor | 0.25 | 1.25 | g |

| | | |
|---|---|---|
| Purified Water | qs | qs |

Process:

Take 300 g of purified water in an appropriate container. Add and dissolve hydroxy propyl β Cyclodextrin under stirring. To this mixture add cineole and winter green oil under stirring and continue stirring until it forms a clear solution. Separately take glycerin and dissolve menthol with slight application of heat (heated to no more than 35° C.) and add this to the main bulk. Make aqueous solutions of other ingredients taking small quantities of water and incorporate in to the main bulk solution under stirring. Incorporate flavor under stirring and check for clarity, pH and make up the volume before filtering the solution through 0.45 micron membrane filter. Fill the liquid in appropriate container closure systems fit with metered dose pump.

### EXAMPLE 8

Oral Cough Suppressant and Decongestant Spray Composition:

This example describes the preparation of an oral cough suppressant and decongestant spray composition in accordance with the invention, which is useful for treating cough and congestion in the throat and relief from nasal congestion. Ingredients for the preparation of the oral cough suppressant and decongestant spray solution are set forth in the table below.

| Ingredient | % quantity | | per 500 mL | |
|---|---|---|---|---|
| Phenylephrine HCl | 3.5 | | 17.5 | g |
| Dextromethorphan HBr | 3.5 | | 17.5 | g |
| Potassium Sorbate | 0.1 | | 0.5 | g |
| Sucralose 25% conc. | 0.5 | | 2.5 | g |
| Xylitol | 7.5 | g | 37.5 | g |
| Propylene Glycol | 30.0 | | 150.0 | g |
| Cineol (eucalyptus oil) | 0.25 | | 1.25 | g |
| Menthol | 0.1 | | 0.5 | g |
| Wintergreen oil | 0.03 | | 0.15 | g |
| Hydroxypropyl β Cyclodextrin | 2.0 | | 10.0 | g |
| Chocolate Fudge | 0.25 | | 1.25 | g |
| Bitter Mask | 0.25 | | 1.25 | g |

| | | |
|---|---|---|
| Purified Water | qs | qs |

Process:

Take 250 g of purified water in an appropriate container. Add and dissolve hydroxy propyl β Cyclodextrin under stirring. To this mixture add cineole and winter green oil under stirring and continue stirring until it forms a clear solution. Separately take propylene glycol 20.0 g and dissolve menthol with slight application of heat (heated to no more than 35° C.) and add this to the main bulk. Dissolve dextromethorphan HBr in propylene glycol 130.0 g with application of heat if needed (heated to no more than 55° C.) and this to main bulk under stirring. Make aqueous solutions of other ingredients taking small quantities of water and incorporate in to the main bulk solution under stirring. Incorporate flavors under stirring and check for clarity, pH and make up the volume before filtering the solution through 0.45 micron membrane filter. Fill the liquid in appropriate container closure systems fit with metered dose pump.

### EXAMPLE 9

Oral Cough Suppressant Spray Composition:

This example describes the preparation of an oral cough suppressant spray composition in accordance with the invention, which is useful for alleviation of cough. Ingredients for the preparation of the oral cough suppressant spray solution are set forth in the table below.

| Ingredient | % quantity | | per 500 mL | |
|---|---|---|---|---|
| Dextromethorphan HBr | 3.0 | | 15.0 | g |
| Potassium Sorbate | 0.1 | | 0.5 | g |
| Sucralose 25% conc. | 0.5 | | 2.5 | g |
| Xylitol | 7.5 | g | 37.5 | g |
| Propylene Glycol | 30.0 | | 150.0 | g |
| Cineol (eucalyptus oil) | 0.25 | | 1.25 | g |
| Menthol | 0.1 | | 0.5 | g |
| Wintergreen oil | 0.03 | | 0.15 | g |
| Hydroxypropyl β Cyclodextrin | 2.0 | | 10.0 | g |
| Cherry flavor | 0.25 | | 1.25 | g |

| | | |
|---|---|---|
| Purified Water | qs | qs |

Process:

Take 250 g of purified water in an appropriate container. Add and dissolve hydroxy propyl β Cyclodextrin under stirring. To this mixture add cineole and winter green oil under stirring and continue stirring until it forms a clear solution. Separately take propylene glycol 20.0 g and dissolve menthol with slight application of heat (heated to no more than 35° C.) and add this to the main bulk. Dissolve dextromethorphan HBr in propylene glycol 130.0 g with application of heat if needed (heated to no more than 55° C.) and add this to main bulk under stirring. Make aqueous solutions of other ingredients taking small quantities of water and incorporate in to the main bulk solution under stirring. Incorporate flavors under stirring and check for clarity, pH and make up the volume before filtering the solution through 0.45 micron membrane filter. Fill the liquid in appropriate container closure systems fit with metered dose pump.

### EXAMPLE 10

Oil and Water Soluble Oral Spray Composition:

This example describes the preparation of oil and water soluble vitamins for oral spray application and is useful as a vitamin supplement. Ingredients for the preparation of the vitamin supplement spray solution are set forth in the table below.

| Ingredient | % quantity | | per 500 mL | |
|---|---|---|---|---|
| Vitamin C Ester | 5.0 | | 25.0 | g |
| Vitamin E (alpha tocopheryl acetate) | 0.1 | | 0.5 | g |
| Vitamin B6 | 2.5 | | 12.5 | g |
| Folic Acid | 1.0 | | 5.0 | g |
| Vitamin D3 (cholecalciferol) | 0.01 | | 0.05 | g |
| Potassium Sorbate | 0.1 | | 0.5 | g |
| Sucralose 25% conc. | 0.5 | | 2.5 | g |
| Xylitol | 7.5 | g | 37.5 | g |
| Propylene Glycol | 5.0 | | 25.0 | g |
| Cineol (eucalyptus oil) | 0.25 | | 1.25 | g |
| Menthol | 0.1 | | 0.5 | g |
| Wintergreen oil | 0.03 | | 0.15 | g |
| Hydroxypropyl β Cyclodextrin | 2.5 | | 12.5 | g |
| Lemon flavor | 0.2 | | 1.0 | g |

| | | |
|---|---|---|
| Purified Water | qs | qs |

Process:

Take 350 g of purified water in an appropriate container. Add and dissolve hydroxy propyl β Cyclodextrin under stirring. To this mixture add cineole and winter green oil under stirring and continue stirring until it forms a clear solution. Separately take propylene glycol 5.0 g and dissolve menthol with slight application of heat (heated to no more than 35° C.) and add this to the main bulk. Dissolve vitamin E and D3 in propylene glycol 20.0 g and add this to main bulk under stirring. Make aqueous solutions of other ingredients taking small quantities of water and incorporate in to the main bulk solution under stirring. Incorporate flavor under stirring and check for clarity, pH and make up the volume before filtering the solution through 0.45 micron membrane filter. Fill the liquid in appropriate container closure systems fit with metered dose pump.

### EXAMPLE 11

Acceptable Recoveries:

Using gas liquid chromatographic run conditions, contents of essential oils were tested. The solutions were passed through 0.45 micron membrane filter prior to injections to remove any suspended insoluble materials.

More than 95 percent assay values of the essential oils indicate that the formulation compositions as indicated in above examples keep essential oils in solution form.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A composition suitable for oral spray or intranasal spray administration which comprises:
a cyclodextrin in an amount of from about 0.1% w/v to about 20% w/v;
at least one essential oil in an amount of from about 0.001% w/v to about 5.0% w/v;
an effective amount of an antimicrobial preservative composition; and
water.

2. The composition of claim 1 further comprising an alcohol co-solvent in an amount of from about 0.2% w/v to about 35% w/v.

3. The composition of claim 1 further comprising a sweetener in an amount of from about 0.1% w/v to about 25% w/v.

4. The composition of claim 1 further comprising a mucoadhesive polymer thickening agent in an amount of from about 0.05% w/v to about 10% w/v.

5. The composition of claim 1 further comprising a flavoring agent in an amount of from about 0.01% w/v to about 2.0% w/v.

6. The composition of claim 1 further comprising a flavor enhancing agent in an amount of from about 0.05% w/v to about 5.0% w/v.

7. The composition of claim 1 further comprising a decongestant, an antitussive, an anticholinergic, an antihistamine, an astringent, an anti-inflammatory steroid composition, a vitamin, a respiratory stimulant, a mucolytic agent, a bronchodilator, a beta-antagonist, an antidiarrheal agent or combinations thereof.

8. The composition of claim 1 wherein the cyclodextrin comprises α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, or combinations thereof.

9. The composition of claim 1 wherein the cyclodextrin comprises hydroxypropyl β-cyclodextrin, sulfobutyl ether β-cyclodextrin, or combinations thereof.

10. The composition of claim 1 wherein the essential oil comprises cineol, thymol, menthol, methyl salicylate wintergreen oil, eucalyptol, carvacrol, camphor, anethole, carvone, eugenol, isoeugenol, limonene, osimen, n-decyl alcohol, citronel, a-salpineol, methyl acetate, citronellyl acetate, methyl eugenol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, star anise, or combinations thereof.

11. The composition of claim 1 wherein the antimicrobial preservative comprises cetyl pyridinium chloride, phenol, benzethonium chloride, butylparaben, methyl paraben, ethyl paraben, propyl paraben, benzalkonium chloride, thimerosal, chlorobutanol, phenylethyl alcohol, benzyl alcohol, potassium sorbate, sodium benzoate, sorbic acid or combinations thereof.

12. The composition of claim 2 wherein the alcohol co-solvent comprises propylene glycol, ethyl alcohol, butyl alcohol, glycerin, hexylene glycol, isopropyl alcohol, polyethylene glycol, polyhydric alcohols, or combinations thereof.

13. The composition of claim 3 wherein the sweetener comprises glucose, fructose, xylitol, sucralose, saccharin, aspartame, acesulfame potassium, cyclamate, neohesperidine DC, thaumatin, alitame, stevioside, or combinations thereof.

14. The composition of claim 1 further comprising an antitussive comprising codeine, hydrocodone, dextromethorphan, dextromethorphan HBr, a demulcent, an expectorant, or combinations thereof.

15. The composition of claim 1 further comprising an anticholinergic comprising atropine, scopolamine, methscopolamine nitrate, glycopyrrolate, benztropine, trihexyphenidyl, or combinations thereof.

16. The composition of claim 1 further comprising a decongestant comprising oxymetazoline HCl, phenylephrine HCl, chlorpheniramine, pseudoephedrine hydrochloride, or combinations thereof.

17. The composition of claim 1 further comprising an antihistamine comprising fexofenadine, chlorpheniramine, chlorpheniramine maleate, cyproheptadine, doxylamine, hydroxyzine, dimenhydrinate, diphenhydramine, doxylamine, meclizine, promethazine, loratidine or combinations thereof.

18. The composition of claim 1 further comprising an astringent comprising zinc gluconate, zinc chloride, zinc acetate, myrrh, acacia, black catechu, carum copticum, tannin, rhatany, alum or combinations thereof.

19. The composition of claim 1 further comprising an anti-inflammatory steroid composition comprising aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, fluticasone propionate, halcinonide, hydrocortisone, icomethasone, meprednisone, methylprednisolone, mometasone paramethasone, mometasone furoate monohydrate, prednisolone, prednisone, tixocortol, triamcinolone, beclomethasone diproprionate, dexamethasone 21-isonicotinate, fluticasone propionate, icomethasone enbutate, tixocortol 21-pivalate, and triamcinolone acetonide, or combinations thereof.

20. The composition of claim 1 further comprising a vitamin comprising Vitamin A, Vitamin B₁, Vitamin B₂, Vitamin B₆, Vitamin B₁₂, folic Acid, Vitamin C, Vitamin D, Vitamin E, Vitamin H, Vitamin K, Vitamin P, or combinations thereof.

21. The composition of claim 1 further comprising a respiratory stimulant comprising progesterone, protriptyline HCl, naloxone HCl, almitrine, doxapram, theophylline, or combinations thereof.

22. The composition of claim 1 further comprising a mucolytic agent comprising trypsin, sodium bicarbonate, acetylcysteine, guaifenesin, or combinations thereof.

23. The composition of claim 1 further comprising a bronchodilator comprising albuterol, albuterol sulfate, epinephrine, ipratropium, isoetharine, isoproterenol, levalbuterol hcl, metaproterenol, pirbuterol acetate, terbutalene, theophylline, or combinations thereof.

24. The composition of claim 1 further comprising a beta-antagonist comprising fenoterol, metaproterenol, procaterol, salbutamol, terbutaline or combinations thereof.

25. The composition of claim 1 further comprising an antidiarrheal agent comprising loperamide hydrochloride, attapulgite, Bismuth subsalicylate or combinations thereof.

26. A metered dose container enclosing the composition of claim 1.

27. A method of oral or nasal care which comprises orally spraying or intranasally spraying to a subject in need of oral or nasal care a composition which comprises:
a cyclodextrin in an amount of from about 0.1% w/v to about 20% w/v;
at least one essential oil in an amount of from about 0.001% w/v to about 5.0% w/v;
an effective amount of an antimicrobial preservative composition; and
water.

28. The method of claim 27 further comprising a thickening agent, a sweetener, an antitussive, an anticholinergic, a decongestant, an antihistamine, an astringent, an anti-inflammatory steroid composition, a vitamin, a respiratory stimulant, a mucolytic agent, a bronchodilator, a beta-antagonist, an antidiarrheal agent, or combinations thereof.
